(19) United States
(12) Patent Application Publication
(10) Pub. No.: US 2025/0339044 A1
(43) Pub. Date: Nov. 6, 2025

(54) BLOOD PRESSURE AND HEART RATE MEASURING DEVICE, PROTECTIVE STRUCTURE, AND BLOOD PRESSURE AND HEART RATE CALCULATING METHOD REQUIRED BY A BLOOD PRESSURE AND HEART RATE MEASURING DEVICE

(71) Applicant: Micro-Nano Development Co., Ltd., Taipei City (TW)

(72) Inventors: Yu-Jie WANG, New Taipei City (TW); Chi-Hung TSENG, New Taipei City (TW); Jiung-Ming TORNG, New Taipei City (TW); Li-Karn WANG, Taipei City (TW)

(73) Assignee: Micro-Nano Development Co., Ltd., Taipei City (TW)

(21) Appl. No.: 18/751,525

(22) Filed: Jun. 24, 2024

(30) Foreign Application Priority Data
May 3, 2024 (TW) 113116530

(51) Int. Cl. A61B 5/024; A61B 5/021

(52) U.S. Cl. CPC A61B 5/02416; A61B 5/02108; A61B 5/02405

(57) ABSTRACT

A blood pressure and heart rate measuring device has two fiber gratings for converting broadband light into a narrowband ray. The reflection wavelength band and the transmission wavelength band of one of the two fiber gratings will shift with the vascular pulsating stress of an object to be measured, which will change the optical power of the narrowband ray. Therefore, the pulse waveform obtained by the blood pressure and heart rate measuring device based on the optical power of the narrowband ray can be used to determine a blood pressure and a heart rate.

### BACKGROUND OF THE INVENTION

This application claims priority for the TW application No. 113116530 filed on 3 May 2024, the content of which is incorporated by reference in its entirely.

### FIELD OF THE INVENTION

The present invention relates to a device and a method for non-invasively measuring a blood pressure and a heart rate using fiber gratings, particularly to a device and a method for measuring the waveform of vascular pulsation based on optical power to calculate a blood pressure and a heart rate.

### DESCRIPTION OF THE RELATED ART

The present methods for measuring a blood pressure and a heart rate are divided into two types, namely an invasive type and a non-invasive type. The invasive method, usually used to measure the blood pressures and the heart rates of patients in intensive care units and operating rooms, involves inserting a needle into the arteries of the subject (such as a patient). However, invasive methods have the risk of infection. Non-invasive blood pressure and heart rate measurement, used by the general public, has relatively convenient and non-invasive advantages. Currently, the most commonly used method for non-invasive blood pressure and heart rate measurement involves the use of an inflatable cuff. The cuff is inflated and then gradually deflated to allow blood to flow through the brachial or radial arteries, thereby generating oscillating pulse wave signals. These signals are detected by a pulsation sensor within the cuff to obtain the values of systolic and diastolic blood pressures. However, since this method requires inflating the cuff prior to measurement, it is not suitable for continuously monitoring a blood pressure and a heart rate.

To overcome the foregoing drawbacks, researchers between 2013 and 2019 proposed using fiber Bragg grating (FBG) sensors for blood pressure and heart rate measurement. Examples include U. Sharath, R. Sukreet, G. Apoorva, and S. Asokan's work, “Blood pressure evaluation using sphygmomanometry assisted by arterial pulse waveform detection by fiber Bragg grating pulse device,” published in the Journal of Biomedical Optics, 18 (6): 067010, 2013; D. Jia, J. Chao, S. Li, H. Zhang, Y. Yan, T. Liu, and Y. Sun's paper, “A fiber Bragg grating sensor for radial artery pulse waveform measurement,” published in IEEE Transactions on Biomedical Engineering, vol. 65, no. 4, pp. 839-846, 2018; and Hiroaki Ishizawa et al.'s study, “Measurement of pulse wave signals and blood pressure by a plastic optical fiber FBG sensor,” published in Sensors, 19 (23): 5088, 2019. These studies measure vascular pulse waveforms (hereinafter referred to as pulse waveforms) by detecting changes in the reflection wavelength of the fiber Bragg grating in order to obtain the values of blood pressures and heart rates.

Additionally, in 2019, U.S. Pat. No. 10,376,160 B2 provided an optical method combined with a blood pressure calibration model for measuring blood pressure. In this patent, a single fiber grating is used to sense vascular pulsation signals, and a Mach-Zehnder interferometer is employed to obtain the pulsation signals as pulse waveforms. These waveforms are then processed by a blood pressure value calculation unit to determine blood pressure values. In 2022, U.S. Pat. No. 11,504,010 B2 provided a wearable device where two sets of fiber gratings are placed on the wrist. This device detects changes in wavelength and estimates blood pressure values in cooperation with a blood pressure estimation model.

However, the current pulse waveforms are generated due to the time-domain variation of the reflection wavelength (commonly known as the Bragg wavelength) of the fiber grating. A wavelength detection instrument is often used to capture the timing variation of the wavelength in order to obtain the pulse waveforms. This detection method is complex and expensive.

### SUMMARY OF THE INVENTION

One of objectives of the present invention is to provide a blood pressure and heart rate measuring device and a method thereof using two fiber gratings, which obtain a pulse waveform by detecting optical power.

One of objectives of the present invention is to provide a protective structure for protecting fiber gratings and fiber optical paths.

One of objectives of the present invention is to provide a blood pressure and heart rate calculation method required by a blood pressure and heart rate measuring device.

According to the present invention, a blood pressure and heart rate measuring device includes at least a broadband light source, an optical coupler, a first fiber grating, a second fiber grating, and a first photo receiver. The broadband light source is configured to provide broadband light. The optical coupler is connected to the broadband light source through a first optical path and configured to receive the broadband light. The first fiber grating, connected to the optical coupler through a second optical path, has a first reflection wavelength band. The first fiber grating is configured to receive a first light ray from the optical coupler through the second optical path and reflect a part of the first light ray whose wavelength band is within the first reflection wavelength band to generate a second light ray that returns to the optical coupler along the second optical path. The first light ray is a part or all of the broadband light. The second fiber grating, connected to the optical coupler through a third optical path, has a second reflection wavelength band. The second fiber grating is configured to receive a third light ray from the optical coupler through the third optical path and allow a part of the third light ray whose wavelength band is not within the second reflection wavelength band to pass to generate a fourth light ray. The third light ray is a part or all of the second light ray. The first photo receiver is connected to the second fiber grating through a fourth optical path and configured to receive the fourth light ray from the second fiber grating through the fourth optical path and generate a first electrical signal of a pulse waveform of vascular pulsation in response to the fourth light ray. The first reflection wavelength band shifts with the vascular pulsating stress of an object to be measured.

According to the present invention, a blood pressure and heart rate measuring device includes at least a broadband light source, a first optical coupler, a first fiber grating, a second optical coupler, a second fiber grating, and a first photo receiver. The broadband light source is configured to provide broadband light. The first optical coupler is connected to the broadband light source through a first optical path and configured to receive the broadband light. The first optical coupler is configured to generate a first light ray and a second light ray in response to the broadband light and respectively transmit the first light ray and the second light ray to a second optical path and a third optical path. The first light ray and the second light ray are parts of the broadband light. The first fiber grating, connected to the first optical coupler through the second optical path and the third optical path, has a first reflection wavelength band. The first fiber grating is configured to reflect a part of the first light ray whose wavelength band is within the first reflection wavelength band to generate a third light ray that returns to the first optical coupler along the second optical path and to reflect a part of the second light ray whose wavelength band is within the first reflection wavelength band to generate a fourth light ray that returns to the first optical coupler along the third optical path. The second optical coupler is configured to receive a fifth light ray from the first optical coupler through a fourth optical path, generate a sixth light ray in response to the fifth light ray, and transmit the sixth light ray to a fifth optical path. The first optical coupler is configured to generate the fifth light ray in response to the third light ray and the fourth light ray, and the sixth light ray is a part or all of the fifth light ray. The second fiber grating, connected to the second optical coupler through a fifth optical path, has a second reflection wavelength band. The second fiber grating is configured to receive the sixth light ray from the second optical coupler through the fifth optical path and reflect a part of the sixth light ray whose wavelength band is within the second reflection wavelength band to generate a seventh light ray that returns to the second optical coupler along the fifth optical path. The first photo receiver is configured to receive an eighth light ray from the second optical coupler through a sixth optical path and generate a first electrical signal of the pulse waveform of vascular pulsation in response to the eighth light ray. The second reflection wavelength band shifts with the vascular pulsating stress of an object to be measured.

According to the present invention, a blood pressure and heart rate measuring device includes at least a laser light source, a first optical coupler, a first fiber grating, an Er-doped fiber, a second optical coupler, a second fiber grating, and a first photo receiver. The laser light source is configured to provide laser light. The first optical coupler is connected to the laser light source through a first optical path and configured to receive the laser light. The first optical coupler is configured to guide the laser light to a second optical path. The first fiber grating, connected to the first optical coupler through the second optical path, has a first reflection wavelength band and a first transmission wavelength band. The laser light from the first optical coupler passes through the first fiber grating. The Er-doped fiber is connected to the first fiber grating and configured to generate broadband light that returns to the first fiber grating in response to the laser light passing through the first fiber grating. A part of the broadband light whose wavelength band is within the first transmission wavelength band passes through the first fiber grating to generate a first light ray that returns to the first optical coupler along the second optical path. The second optical coupler is configured to receive a second light ray from the first optical coupler through a third optical path, generate a third light ray in response to the second light ray, and transmit the third light ray to a fourth optical path. The second light ray is a part or all of the first light ray and the third light ray is a part or all of the second light ray. The second fiber grating, connected to the second optical coupler through the fourth optical path, has a second reflection wavelength band and a second transmission wavelength band. The second fiber grating is configured to receive the third light ray from the second optical coupler through the fourth optical path and reflect a part of the third light ray whose wavelength band is within the second reflection wavelength band to generate a fourth light ray that returns to the second optical coupler along the fourth optical path. The first photo receiver is configured to receive a fifth light ray from the second optical coupler through a fifth optical path and generate a first electrical signal of the pulse waveform of vascular pulsation in response to the fifth light ray. The fifth light ray is a part or all of the fourth light ray. The second reflection wavelength band shifts with the vascular pulsating stress of an object to be measured.

According to the present invention, a blood pressure and heart rate measuring device includes at least a broadband light source, a first fiber grating, an optical coupler, a second fiber grating, and a first photo receiver. The broadband light source is configured to provide broadband light. The first fiber grating, connected to the broadband light source through a first optical path and configured to receive the broadband light, has a first reflection wavelength band and a first transmission wavelength band. A part of the broadband light whose wavelength band is within the first transmission wavelength band passes through the first fiber grating to form a first light ray. The optical coupler is connected to the first fiber grating through a second optical path and configured to receive the first light ray. The second fiber grating, connected to the optical coupler through a third optical path, has a second reflection wavelength band and a second transmission wavelength band. The second fiber grating is configured to receive a second light ray from the optical coupler through the third optical path and reflect a part of the second light ray whose wavelength band is within the second reflection wavelength band to generate a third light ray that returns to the optical coupler along the third optical path. The second light ray is a part or all of the first light ray. The first photo receiver is connected to the optical coupler through a fourth optical path and configured to receive a fourth light ray from the optical coupler through the fourth optical path and generate a first electrical signal of the pulse waveform of vascular pulsation in response to the fourth light ray. The fourth light ray is a part or all of the third light ray. The second reflection wavelength band shifts with the vascular pulsating stress of an object to be measured.

According to the present invention, a blood pressure and heart rate measuring device includes at least a broadband light source, an optical coupler, a first fiber grating, a second fiber grating, and a first photo receiver. The broadband light source is configured to provide broadband light. The optical coupler is connected to the broadband light source through a first optical path and configured to receive the broadband light. The first fiber grating, connected to the optical coupler through a second optical path, has a first reflection wavelength band and a first transmission wavelength band. The first fiber grating is configured to receive a first light ray from the optical coupler through the second optical path and reflect a part of the first light ray whose wavelength band is within the first reflection wavelength band to generate a second light ray that returns to the optical coupler along the second optical path. The first light ray is a part or all of the broadband light. A part of the first light ray whose wavelength band is within the first transmission wavelength band passes through the first fiber grating to form a third light ray. The second fiber grating, connected to the first fiber grating through a third optical path, has a second reflection wavelength band and a second transmission wavelength band. The second fiber grating is configured to receive the third light ray from the first fiber grating through the third optical path and reflect a part of the third light ray whose wavelength band is within the second reflection wavelength band to generate a fourth light ray that returns to the optical coupler along the third optical path, the first fiber grating, and the second optical path. The first photo receiver is connected to the optical coupler through a fourth optical path and configured to receive a fifth light ray from the optical coupler through the fourth optical path and generate a first electrical signal of the pulse waveform of vascular pulsation in response to the fifth light ray. The fifth light ray is parts or all of the second light ray and the fourth light ray. The first reflection wavelength band or the second reflection wavelength band shifts with the vascular pulsating stress of an object to be measured.

According to the present invention, a protective structure for protecting a fiber grating and a fiber optical path includes a stretchable material and a tube that is of plastic type or of rubber type. The stretchable material is configured to cover the fiber grating, a first fiber optical path, a second fiber optical path, and the first portion of a third fiber optical path. The third fiber optical path has a first portion inside the stretchable material, and a second portion outside the stretchable material. One end of the first fiber optical path is connected to the fiber grating and another end of the first fiber optical path is connected to the first portion of the third fiber optical path. The second fiber optical path has a second portion outside the stretchable material. The tube has a first portion and a second portion. The first portion of the tube, located inside the stretchable material, covers the first portion of the third fiber optical path. The second portion of the tube, located outside the stretchable material, covers the second portion of the third fiber optical path. A first light ray, entering the interior of the stretchable material from the third fiber optical path, is emitted to the fiber grating through the first fiber optical path. A part of the first light ray whose wavelength band is within the reflection wavelength band of the fiber grating is reflected to form a second light ray that returns to the first fiber optical path and then the third fiber optical path. The second light ray is received by a photo receiver to generate an electrical signal of the pulse waveform of vascular pulsation.

Below, the embodiments are described in detail in cooperation with the drawings to make easily understood the technical contents, characteristics and accomplishments of the present invention.

### BRIEF DESCRIPTION OF THE DRAWINGS

FIG. 1 is a schematic diagram showing a blood pressure and heart rate measuring device according to a first embodiment of the present invention;

FIG. 2 is a schematic diagram showing the first reflection wavelength band RB1 of a first fiber grating 15a and the second transmission wavelength band TB2 of a second fiber grating 15b when not affected by vascular pulsating stress;

FIG. 3 is a schematic diagram showing the first reflection wavelength band RB1 of a first fiber grating 15a and the second transmission wavelength band TB2 of a second fiber grating 15b when affected by vascular pulsating stress;

FIG. 4 is a schematic diagram showing a blood pressure and heart rate measuring device according to a second embodiment of the present invention;

FIG. 5 is a schematic diagram showing a blood pressure and heart rate measuring device according to a third embodiment of the present invention;

FIG. 6 is a schematic diagram showing a blood pressure and heart rate measuring device according to a fourth embodiment of the present invention;

FIG. 7 is a schematic diagram showing a blood pressure and heart rate measuring device according to a fifth embodiment of the present invention;

FIG. 8 is a schematic diagram showing a blood pressure and heart rate measuring device according to a sixth embodiment of the present invention;

FIG. 9 is a schematic diagram showing a blood pressure and heart rate measuring device according to a seventh embodiment of the present invention;

FIG. 10 is a schematic diagram showing a blood pressure and heart rate measuring device according to an eighth embodiment of the present invention;

FIG. 11 is a schematic diagram showing a blood pressure and heart rate measuring device according to a ninth embodiment of the present invention;

FIG. 12 is a schematic diagram showing a blood pressure and heart rate measuring device according to a tenth embodiment of the present invention;

FIG. 13 is a schematic diagram showing a pulse waveform signal obtained by a blood pressure and heart rate measuring device;

FIG. 14 is an enlarged view of a part of the pulse waveform signal in FIG. 13;

FIG. 15 is a schematic diagram showing a protective structure for protecting a fiber grating and a fiber optical path adjacent thereto;

FIG. 16 is a schematic diagram showing the arm belt of a blood pressure and heart rate measuring device; and

FIG. 17 is a cross-sectional view of an arm belt in FIG. 16 that surrounds an arm.

### DETAILED DESCRIPTION OF THE INVENTION

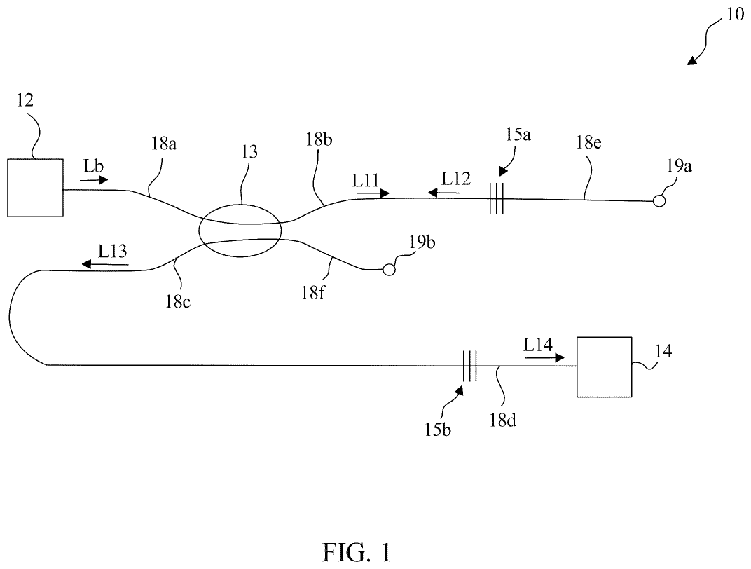

FIG. 1 is a schematic diagram showing a blood pressure and heart rate measuring device according to a first embodiment of the present invention. As shown in FIG. 1, a blood pressure and heart rate measuring device 10 includes at least a broadband light source 12, an optical coupler 13, a first fiber grating 15a, a second fiber grating 15b, and a first photo receiver 14. The broadband light source 12 provides broadband light Lb for the optical coupler 13 through a first optical path 18a. The optical coupler 13 generates a first light ray L11 in response to the broadband light Lb and transmits the first light ray L11 to the first fiber grating 15a through a second optical path 18b. The first light ray L11 is a part or all of the broadband light Lb. The first fiber grating 15a has a first reflection wavelength band RB1 and a first transmission wavelength band TB1. The part of the first light beam L11 whose wavelength band is within the first reflection wavelength band RB1 will be reflected by the first fiber grating 15a to generate a second light ray L12 that returns to the optical coupler 13 along the second optical path 18b. After receiving the second light ray L12, the optical coupler 13 generates a third light ray L13 and guides it to the second fiber grating 15b along a third optical path 18c. The second fiber grating 15b has a second reflection wavelength band RB2 and a second transmission wavelength band TB2. The part of the third light ray L13 whose wavelength band is not within the second reflection wavelength band RB2 (i.e., within the second transmission wavelength band TB2) passes through the fiber grating 15b to form a fourth light ray L14. The first photo receiver 14 receives the fourth light ray L14 from the second fiber grating 15b through a fourth optical path 18d and generates a first electrical signal of the pulse waveform of vascular pulsation based on the optical power of the fourth light ray L14. The first electrical signal is converted into a current signal or voltage signal.

In FIG. 1, the first fiber grating 15a is adjacent to the surface of the object to be measured (such as human skin). Thus, the first fiber grating 15a will shift the first reflection wavelength band RB1 and the first transmission wavelength band TB1 due to vascular pulsating stress. The second fiber grating 15b is far away from the surface of the object to be measured. The second fiber grating 15b is less or not affected by the vascular pulsating stress. Therefore, the second reflection wavelength band RB2 and the second transmission wavelength band TB2 of the second fiber grating 15b will only shift slightly or not shift at all.

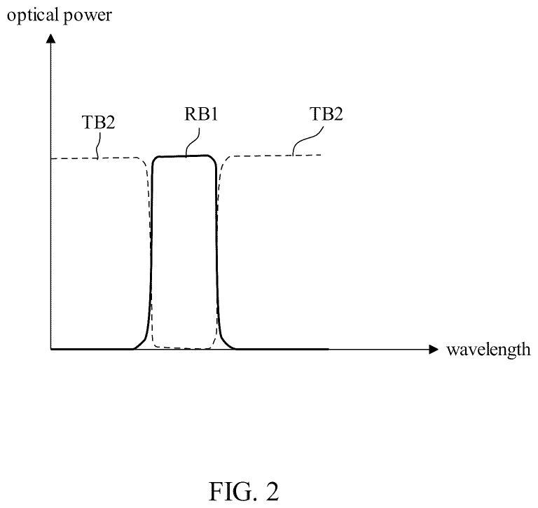

FIG. 2 is a schematic diagram showing the first reflection wavelength band RB1 of a first fiber grating 15a and the second transmission wavelength band TB2 of a second fiber grating 15b when not affected by vascular pulsating stress. When the first fiber grating 15a and the second fiber grating 15b have the same reflection wavelength band (that is, the first reflection wavelength band RB1 and the second reflection wavelength band RB2 are identical, such that the first reflection wavelength band RB1 and the second transmission wavelength band TB2 are complementary, as shown in FIG. 2). If the first fiber grating 15a does not bear any vascular pulsating stress, the wavelength band of the third light ray L13 is almost completely within the second reflection wavelength band RB2 of the second fiber grating 15b. In other words, the optical power of the fourth light ray L14 received by the first photo receiver 14 is very small or almost zero.

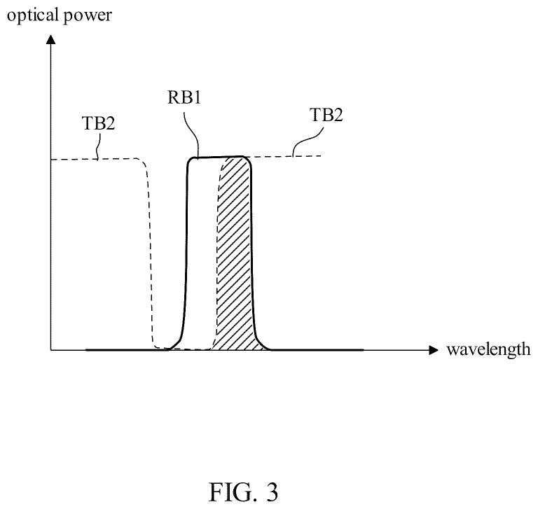

FIG. 3 is a schematic diagram showing the first reflection wavelength band RB1 of a first fiber grating 15a and the second transmission wavelength band TB2 of a second fiber grating 15b when affected by vascular pulsating stress. When the first fiber grating 15a bears vascular pulsating stress such that the first reflection wavelength band RB1 shifts to the right, the first reflection wavelength band RB1 of the first fiber grating 15a will partially overlap the second transmission wavelength band TB2 of the second fiber grating 15b, as shown in the slash region of FIG. 3. The size of the slash region is directly proportional to the optical power of the fourth light ray L14 passing through the second fiber grating 15b. If the first fiber grating 15a bears lower vascular pulsating stress, the area of the slash region becomes smaller, the optical power of the fourth light ray L14 becomes lower, and thus the electrical signal outputted by the first photo receiver 14 becomes smaller. On the contrary, if the first fiber grating 15a bears higher vascular pulsating stress, the area of the slash region becomes larger, the optical power of the fourth light ray L14 becomes higher, and thus the electrical signal outputted by the first photo receiver 14 becomes larger. That is to say, when the vascular pulsating stress applied to the first fiber grating 15a fluctuates, the electrical signal outputted by the first photo receiver 14 fluctuates, thereby forming the pulse waveform of vascular pulsation.

In FIG. 1, the blood pressure and heart rate measuring device 10 further includes a fifth optical path 18e and a sixth optical path 18f respectively connected to the first fiber grating 15a and the optical coupler 13. In order to avoid Fresnel reflections at the end surfaces 19a and 19b of the fifth optical path 18e and the sixth optical path 18f, there are several ways to avoid reflecting light rays back into the optical paths: (1) coating anti-reflective materials on the end surfaces 19a and 19b; (2) designing the end faces 19a and 19b to have an inclination angle greater than 3 degrees; and (3) designing the optical paths near the end faces 19a and 19b to have curled shapes with small radiuses of curvature to produce the bending loss of light.

The first optical path 18a, the second optical path 18b, the third optical path 18c, the fourth optical path 18d, the fifth optical path 18e, and the sixth optical path 18f in FIG. 1 can be fiber optical paths, waveguide optical paths, or fiber and waveguide mixed optical paths.

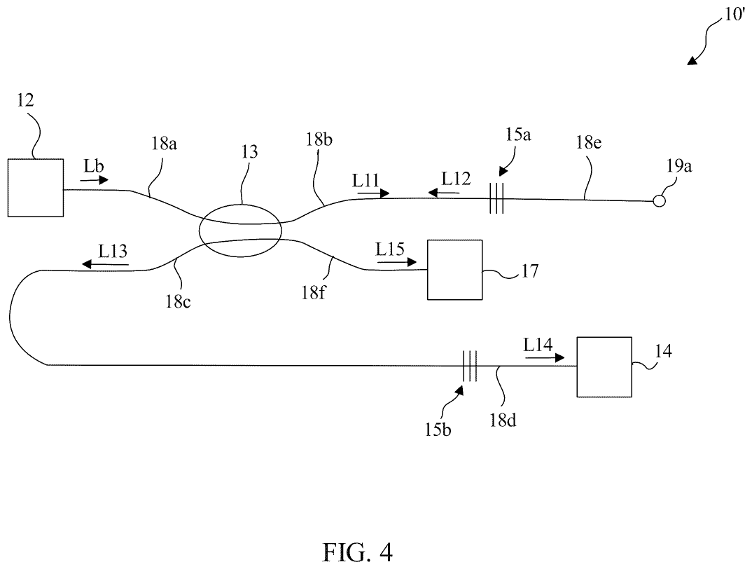

FIG. 4 is a schematic diagram showing a blood pressure and heart rate measuring device according to a second embodiment of the present invention. The blood pressure and heart rate measuring device of FIG. 1 is almost the same as that of FIG. 4. Their difference is that the blood pressure and heart rate measuring device 10′ of FIG. 4 further includes a second photo receiver 17 connected to the optical coupler 13 through a sixth optical path 18f. When the optical power of the broadband light source 12 is unstable, the optical power of the fourth light ray L14 received by the first photo receiver 14 will also be unstable, which may distort the finally obtained pulse waveform. The blood pressure and heart rate measuring device 10′ employs the second photo receiver 17 to improve the problem. Specifically, after the optical coupler 13 receives the broadband light Lb, the optical coupler 13 will generate a fifth light ray L15 and transmit the fifth light ray L15 to the second photo receiver 17 through the sixth optical path 18f. The second photo receiver 17 generates a second electrical signal in response to the fifth light ray L15. Since the first electrical signal and the second electrical signal both include unstable components of the broadband light source 12, the blood pressure and heart rate measuring device 10′ can cancel out the unstable components when dividing the first electrical signal by the second electrical signal. In other words, the pulsating waveform signal obtained by dividing the first electrical signal by the second electrical signal excludes the unstable component and will not be affected by the instability of the broadband light source 12.

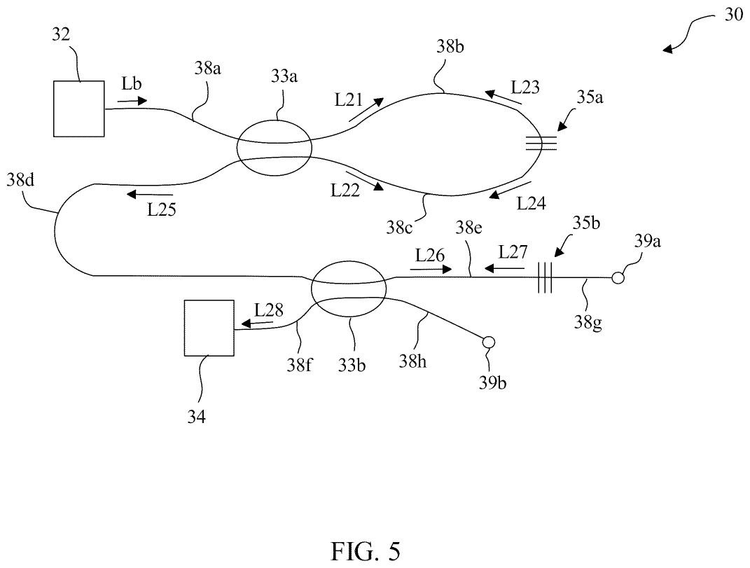

FIG. 5 is a schematic diagram showing a blood pressure and heart rate measuring device according to a third embodiment of the present invention. The blood pressure and heart rate measuring device 30 of FIG. 5 includes at least a broadband light source 32, a first optical coupler 33a, a second optical coupler 33b, a first fiber grating 35a, a second fiber grating 35b, a first photo receiver 34. The broadband light source 32 provides broadband light Lb for the first photo coupler 33a through a first optical path 38a. The first optical coupler 33a generates a first light ray L21 and a second light ray L22 in response to the broadband light Lb and transmits the first light ray L21 and the second light ray L22 to the first fiber grating 35a through a second optical path 38b and a third optical path 38c, respectively. The first light ray L21 and the second light ray L22 is parts or all of the broadband light Lb. The first fiber grating 35a has a first reflection wavelength band RB1 and a first transmission wavelength band TB1. The part of the first light ray L21 whose wavelength band is within the first reflection wavelength band RB1 will be reflected by the first fiber grating 35a to generate a third light ray L23 that returns to the first optical coupler 33a along the second optical path 38b. The part of the second light ray L22 whose wavelength band is within the first reflection wavelength band RB1 will be reflected by the first fiber grating 35a to generate a fourth light ray L24 that returns to the first optical coupler 33a along the third optical path 38c. After receiving the third light ray L23 and the fourth light ray L24, the first optical coupler 33a generates a fifth light ray L25 and provides it for the second optical coupler 33b along a fourth optical path 38d, where the fifth light ray L25 is a part or all of the sum of the third light ray L23 and the fourth light ray L24. The second optical coupler 33b generates a sixth light ray L26 in response to the fifth light ray L25 and provides the sixth light ray L26 for the second fiber grating 35b through a fifth optical path 38e. The second fiber grating 35b has a second reflection wavelength band RB2 and a second transmission wavelength band TB2. The part of the sixth light ray L26 whose wavelength band is within the second reflection wavelength band RB2 is reflected by the second fiber grating 35b to generate a seventh light ray L27 that returns to the second optical coupler 33b along the fifth optical path 38e. The second optical coupler 33b generates an eighth light ray L28 in response to the seventh light ray L27 and provides the eighth light ray L28 for the first photo receiver 34 through a sixth optical path 38f. The first photo receiver 34 generates a first electrical signal of the pulse waveform of vascular pulsation based on the optical power of the eighth light ray L28, wherein the first electrical signal is converted into a current signal or a voltage signal. The eighth light ray L28 has a wavelength band within the overlapping wavelength band of the first reflection wavelength band RB1 and the second reflection wavelength band RB2.

In FIG. 5, the second fiber grating 35b is adjacent to the surface of an object to be measured (such as human skin). Thus, the second fiber grating 35b will shift the second reflection wavelength band RB2 and the second transmission wavelength band TB2 due to the vascular pulsating stress. Since the first fiber grating 35a is far away from the surface of the object to be measured, the first fiber grating 35a is less or not affected by the vascular pulsating stress. Therefore, the first reflection wavelength band RB1 and the first transmission wavelength band TB1 of the first fiber grating 35a will only shift slightly or not shift at all. When the second fiber grating 35b bears the vascular pulsating stress, the second reflection wavelength band RB2 shifts with vascular pulsation, which changes the area of the overlapping wavelength band of the first reflection wavelength band RB1 and the second reflection wavelength band RB2. In other words, the optical power of the eighth light ray L28 received by the first photo receiver 34 changes with the vascular pulsation. Thus, the optical power received by the first photo receiver 34 presents a pulse waveform signal.

In FIG. 5, the blood pressure and heart rate measuring device 30 further includes a seventh optical path 38g and an eighth optical path 38h respectively connected to the second fiber grating 35b and the second optical coupler 33b. In order to avoid Fresnel reflections at the end surfaces 39a and 39b of the seventh optical path 38g and the eighth optical path 38h, there are several ways to avoid reflecting light rays back into the optical paths: (1) coating anti-reflective materials on the end surfaces 39a and 39b; (2) designing the end faces 39a and 39b to have an inclination angle greater than 3 degrees; and (3) designing the optical paths near the end faces 39a and 39b to have curled shapes with small radiuses of curvature to produce the bending loss of light.

The first optical path 38a, the second optical path 38b, the third optical path 38c, the fourth optical path 38d, the fifth optical path 38e, the sixth optical path 38f, the seventh optical path 38g, and the eighth optical path 38h in FIG. 5 can be fiber optical paths, waveguide optical paths, or fiber and waveguide mixed optical paths.

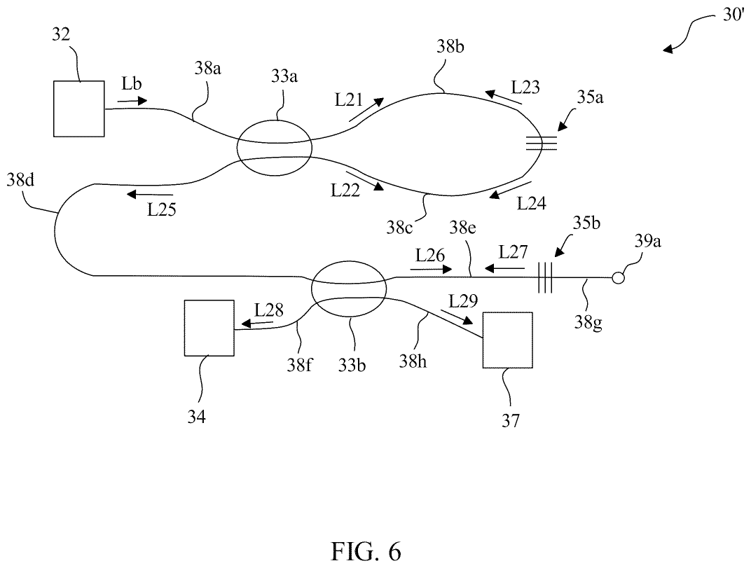

FIG. 6 is a schematic diagram showing a blood pressure and heart rate measuring device according to a fourth embodiment of the present invention. The blood pressure and heart rate measuring device of FIG. 5 is almost the same as that of FIG. 6. Their difference is that the blood pressure and heart rate measuring device 30′ of FIG. 6 further includes a second photo receiver 37 connected to the second optical coupler 33b through an eighth optical path 38h. When the optical power of the broadband light source 32 is unstable, the optical power of the eighth light ray L28 received by the first photo receiver 34 will also be unstable, which may distort the finally obtained pulse waveform. The blood pressure and heart rate measuring device 30′ employs the second photo receiver 37 to improve the problem. Specifically, after the second optical coupler 33b receives the fifth light ray L25, the second optical coupler 33b will generate a ninth light ray L29 and transmit the ninth light ray L29 to the second photo receiver 37 through the eighth optical path 38h. The second photo receiver 37 generates a second electrical signal in response to the ninth light ray L29. Since the first electrical signal and the second electrical signal both include unstable components of the broadband light source 32, the blood pressure and heart rate measuring device 30′ can cancel out the unstable components when dividing the first electrical signal by the second electrical signal. In other words, the pulsating waveform signal obtained by dividing the first electrical signal by the second electrical signal excludes the unstable component and will not be affected by the instability of the broadband light source 32.

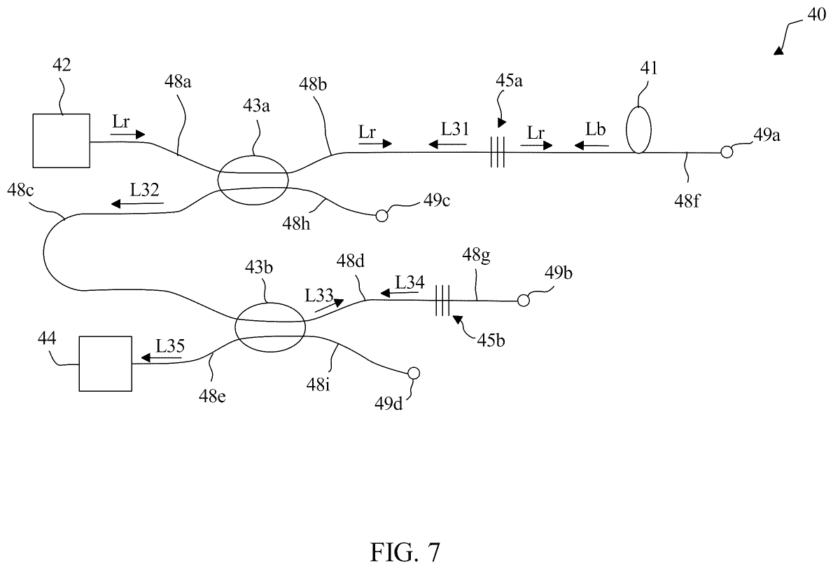

FIG. 7 is a schematic diagram showing a blood pressure and heart rate measuring device according to a fifth embodiment of the present invention. The blood pressure and heart rate measuring device 40 of FIG. 7 includes at least an erbium (Er)-doped fiber 41, a laser light source 42, a first optical coupler 43a, a second optical coupler 43b, a first photo receiver 44, a first fiber grating 45a, and a second fiber grating 45b. The laser light source 42 provides laser light Lr for the first optical coupler 43a through a first optical path 48a. The first optical coupler 43a has a wavelength division multiplexing function. The first optical coupler 43a guides the laser light Lr to a second optical path 48b, so that the laser light Lr is emitted to the first fiber grating 45a through the second optical path 48b. The first fiber grating 45a has a first reflection wavelength band RB1 and a first transmission wavelength band TB1. The laser light Lr passes through the first fiber grating 45a and enters the erbium-doped fiber 41. The erbium-doped fiber 41 generates broadband light Lb in response to the laser light Lr and transmits the broadband light Lb back to the first fiber grating 45a. The central wavelength of the broadband light Lb ranges between 1500 nm and 1590 nm. The part of the broadband light Lb whose wavelength band is within the first transmission wavelength band TB1 will pass through the first fiber grating 45a to generate a first light ray L31. The first light ray L31 is emitted to the first optical coupler 43a along the second optical path 48b. The first optical coupler 43a generates a second light ray L32 in response to the first light ray L31. The second light L32 is provided to the second optical coupler 43b through a third optical path 48c. The second optical coupler 43b generates a third light ray L33 in response to the second light ray L32 and transmits the third light ray L33 to the second fiber grating 45b along a fourth optical path 48d. The second fiber grating 45b has a second reflection wavelength band RB2 and a second transmission wavelength band TB2. The part of the third light ray L33 whose wavelength band is within the second reflection wavelength band RB2 is reflected by the second fiber grating 45b to generate a fourth light ray L34. The fourth light ray L34 returns to the second optical coupler 43b along the fourth optical path 48d. The second optical coupler 43b generates a fifth light ray L35 in response to the fourth light ray L34 and transmits the fifth light ray L35 to the first photo receiver 44 along a fifth optical path 48e. The first photo receiver 44 generates a first electrical signal of the pulse waveform of vascular pulsation based on the optical power of the fifth light ray L35, wherein the first electrical signal is converted into a current signal or a voltage signal. The fifth light ray L35 has a wavelength band within the overlapping wavelength band of the first transmission wavelength band TB1 and the second reflection wavelength band RB2.

In FIG. 7, the second fiber grating 45b is adjacent to the surface of an object to be measured (such as human skin). Thus, the second fiber grating 45b will shift the second reflection wavelength band RB2 and the second transmission wavelength band TB2 due to the vascular pulsating stress. Since the first fiber grating 45a is far away from the surface of the object to be measured, the first fiber grating 45a is less or not affected by the vascular pulsating stress. Therefore, the first reflection wavelength band RB1 and the first transmission wavelength band TB1 of the first fiber grating 45a will only shift slightly or not shift at all. When the second fiber grating 45b bears the vascular pulsating stress, the second reflection wavelength band RB2 shifts with vascular pulsation, which changes the area of the overlapping wavelength band of the first transmission wavelength band TB1 and the second reflection wavelength band RB2. In other words, the optical power of the fifth light ray L35 received by the first photo receiver 44 changes with the vascular pulsation. Thus, the optical power received by the first photo receiver 44 presents a pulse waveform signal.

In FIG. 7, the blood pressure and heart rate measuring device 40 further includes a sixth optical path 48f, a seventh optical path 48g, an eighth optical path 48h, and a ninth optical path 48i respectively connected to the Er-doped fiber 41, the second fiber grating 45b, the first optical coupler 43a, and the second optical coupler 43b. In order to avoid Fresnel reflections at the end surfaces 49a, 49b, 49c, and 49d of the sixth optical path 48f, the seventh optical path 48g, the eighth optical path 48h, and the ninth optical path 49i, there are several ways to avoid reflecting light rays back into the optical paths: (1) coating anti-reflective materials on the end surfaces 49a, 49b, 49c, and 49d; (2) designing the end faces 49a, 49b, 49c, and 49d to have an inclination angle greater than 3 degrees; and (3) designing the optical paths near the end faces 49a, 49b, 49c, and 49d to have curled shapes with small radiuses of curvature to produce the bending loss of light.

The first optical path 48a, the second optical path 48b, the third optical path 48c, the fourth optical path 48d, the fifth optical path 48e, the sixth optical path 48f, the seventh optical path 48g, the eighth optical path 48h, and the ninth optical 48i in FIG. 7 can be fiber optical paths, waveguide optical paths, or fiber and waveguide mixed optical paths.

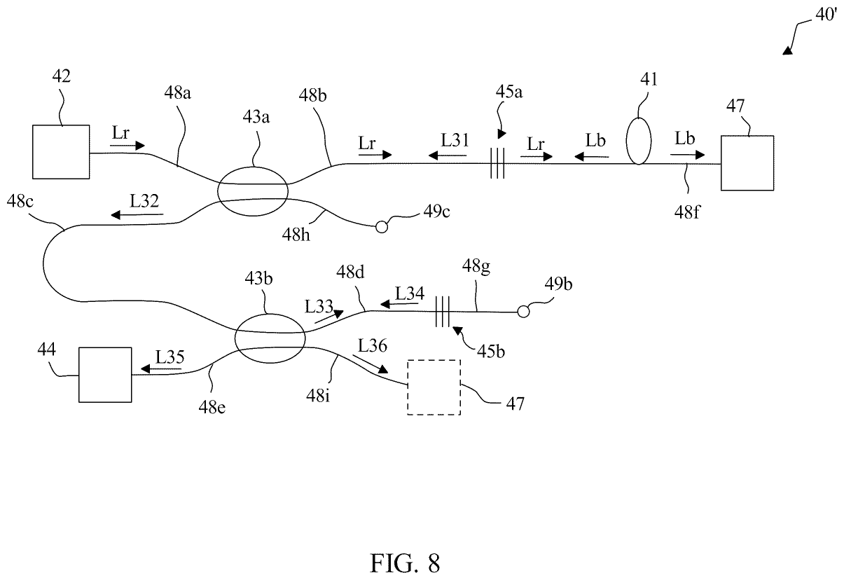

FIG. 8 is a schematic diagram showing a blood pressure and heart rate measuring device according to a sixth embodiment of the present invention. The blood pressure and heart rate measuring device of FIG. 7 is almost the same as that of FIG. 8. Their difference is that the blood pressure and heart rate measuring device 40′ of FIG. 8 further includes a second photo receiver 47 connected to the Er-doped fiber 41 through the sixth optical path 48f or connected to the second optical coupler 43b through the ninth optical path 48i. When the optical power of the Er-doped fiber 41 is unstable, the optical power of the fifth light ray L35 received by the first photo receiver 44 will also be unstable, which may distort the finally obtained pulse waveform.

The blood pressure and heart rate measuring device 40′ employs the second photo receiver 47 to improve the problem. Specifically, after the second photo receiver 47 receives a broadband light Lb through the sixth optical path 48f or receives the sixth light ray L36 through the ninth optical path 48i, the second photo receiver 47 will generate a second electrical signal in response to the broadband light Lb or the sixth light ray L36. Since the first electrical signal and the second electrical signal both include unstable components of the broadband light Lb, the blood pressure and heart rate measuring device 40′ can cancel out the unstable components when dividing the first electrical signal by the second electrical signal. In other words, the pulsating waveform signal obtained by dividing the first electrical signal by the second electrical signal excludes the unstable component and will not be affected by the instability of the broadband light Lb.

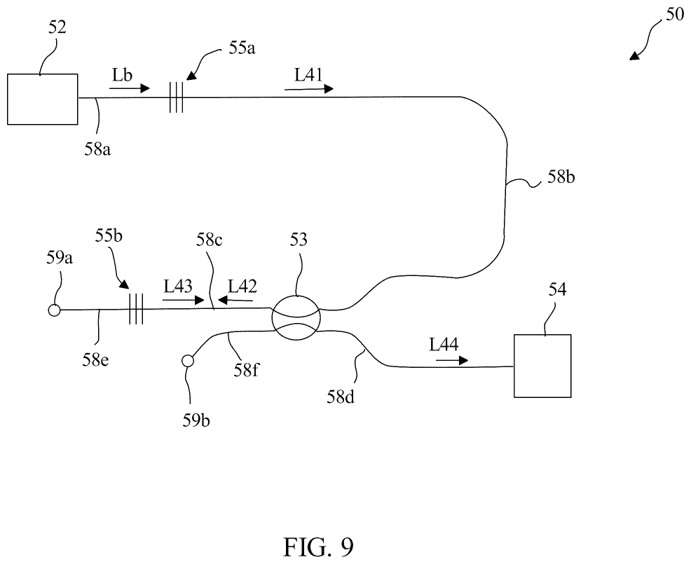

FIG. 9 is a schematic diagram showing a blood pressure and heart rate measuring device according to a seventh embodiment of the present invention. The blood pressure and heart rate measuring device 50 of FIG. 9 includes at least a broadband light source 52, an optical coupler 53, a first photo receiver 54, a first fiber grating 55a, and a second fiber grating 55b. The broadband light source 52 provides broadband light Lb for the first fiber grating 55a through a first optical path 58a. The first fiber grating 55a has a first reflection wavelength band RB1 and a first transmission wavelength band TB1. The part of the broadband light Lb whose wavelength band is within the first transmission wavelength band TB1 will pass through the first fiber grating 55a to generate a first light ray L41. The first light ray L41 enters the optical coupler 53 along a second optical path 58b. The optical coupler 53 generates a second light ray L42 in response to the first light ray L41 and transmits the second light ray L42 to the second fiber grating 55b through a third optical path 58c, where the second light ray L42 is a part or all of the first light ray L41. The second fiber grating 55b has a second reflection wavelength band RB2 and a second transmission wavelength band TB2. The part of the second light ray L42 whose wavelength band is within the second reflection wavelength band RB2 is reflected by the second fiber grating 55b to generate a third light ray L43 that returns to the optical coupler 53 along the third optical path 58c. The optical coupler 53 generates a fourth light ray L44 in response to the third light ray L43 and provides the fourth light ray L44 for the first photo receiver 54 through a fourth optical path 58d. The first photo receiver 54 generates a first electrical signal of the pulse waveform of vascular pulsation based on the optical power of the fourth light ray L44, wherein the first electrical signal is converted into a current signal or a voltage signal.

In FIG. 9, the second fiber grating 55b is adjacent to the surface of an object to be measured (such as human skin). Thus, the second fiber grating 55b will shift the second reflection wavelength band RB2 and the second transmission wavelength band TB2 due to the vascular pulsating stress. Since the first fiber grating 55a is far away from the surface of the object to be measured, the first fiber grating 55a is less or not affected by the vascular pulsating stress. Therefore, the first reflection wavelength band RB1 and the first transmission wavelength band TB1 of the first fiber grating 55a will only shift slightly or not shift at all. When the second fiber grating 55b bears the vascular pulsating stress, the second reflection wavelength band RB2 shifts with vascular pulsation, which changes the area of the overlapping wavelength band of the first transmission wavelength band TB1 and the second reflection wavelength band RB2. In other words, the optical power of the fourth light ray L44 received by the first photo receiver 54 changes with the vascular pulsation. Thus, the optical power received by the first photo receiver 54 presents a pulse waveform signal.

In FIG. 9, the blood pressure and heart rate measuring device 50 further includes a fifth optical path 58e and a sixth optical path 58f respectively connected to the fiber grating 55b and the optical coupler 53. In order to avoid Fresnel reflections at the end surfaces 59a and 59b of the fifth optical path 58e and the sixth optical path 58f, there are several ways to avoid reflecting light rays back into the optical paths: (1) coating anti-reflective materials on the end surfaces 59a and 59b; (2) designing the end faces 59a and 59b to have an inclination angle greater than 3 degrees; and (3) designing the optical paths near the end faces 59a and 59b to have curled shapes with small radiuses of curvature to produce the bending loss of light.

The first optical path 58a, the second optical path 58b, the third optical path 58c, the fourth optical path 58d, the fifth optical path 58e, and the sixth optical path 58f in FIG. 9 can be fiber optical paths, waveguide optical paths, or fiber and waveguide mixed optical paths.

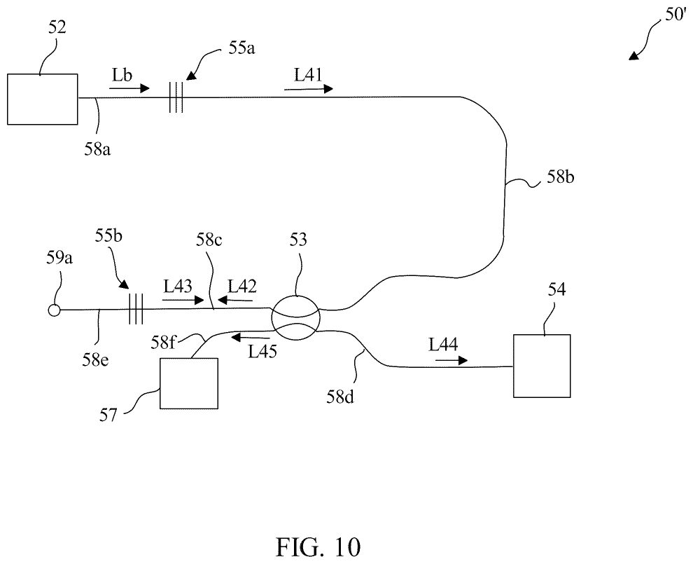

FIG. 10 is a schematic diagram showing a blood pressure and heart rate measuring device according to an eighth embodiment of the present invention. The blood pressure and heart rate measuring device of FIG. 9 is almost the same as that of FIG. 10. Their difference is that the blood pressure and heart rate measuring device 50′ of FIG. 10 further includes a second photo receiver 57 connected to the optical coupler 53 through the sixth optical path 58f. When the optical power of the broadband light source 52 is unstable, the optical power of the fourth light ray L44 received by the first photo receiver 54 will also be unstable, which may distort the finally obtained pulse waveform. The blood pressure and heart rate measuring device 50′ employs the second photo receiver 57 to improve the problem. Specifically, after the optical coupler 53 receives the first light ray L41, the optical coupler 53 will generate a fifth light ray L45 and transmits the fifth light ray L45 to the second photo receiver 57 through the sixth optical path 58f. The second photo receiver 57 generates a second electrical signal in response to the fifth light ray L45. Since the first electrical signal and the second electrical signal both include unstable components of the broadband light source 52, the blood pressure and heart rate measuring device 50′ can cancel out the unstable components when dividing the first electrical signal by the second electrical signal. In other words, the pulsating waveform signal obtained by dividing the first electrical signal by the second electrical signal excludes the unstable component and will not be affected by the instability of the broadband light source 52.

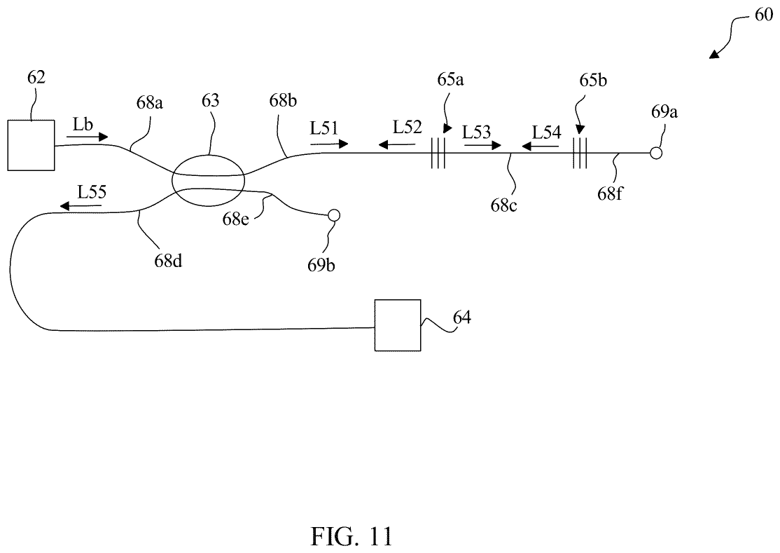

FIG. 11 is a schematic diagram showing a blood pressure and heart rate measuring device according to a ninth embodiment of the present invention. The blood pressure and heart rate measuring device 60 of FIG. 11 includes at least a broadband light source 62, an optical coupler 63, a first photo receiver 64, a first fiber grating 65a, and a second fiber grating 65b. The broadband light source 62 provides broadband light Lb for the optical coupler 63 through a first optical path 68a. The optical coupler 63 generates a first light ray L51 in response to the broadband light Lb and transmits the first light ray L51 to the first fiber grating 65a through a second optical path 68b, where the first light ray L51 is a part or all of the broadband light Lb. The first fiber grating 65a has a first reflection wavelength band RB1 and a first transmission wavelength band TB1. The part of the first light ray L51 whose wavelength band is within the first reflection wavelength band RB1 will be reflected by the first fiber grating 65a to generate a second light ray L52 that returns to the optical coupler 63 along the second optical path 68b. The part of the first light ray L51 whose wavelength band is within the first transmission wavelength band TB1 will pass through the first fiber grating 65a to generate a third light ray L53 that is emitted to the second fiber grating 65b along a third optical path 68c. The second fiber grating 65b has a second reflection wavelength band RB2 and a second transmission wavelength band TB2. The part of the third light ray L53 whose wavelength band is within the second reflection wavelength band RB2 is reflected by the second fiber grating 65b to form a fourth light ray L54 that returns to the optical coupler 63 through the third optical path 68c, the first fiber grating 65a, and the second optical path 68b. After receiving the second light ray L52 and the fourth light ray L54, the optical coupler 63 generates a fifth light ray L55 and provides it for the first photo receiver 64 along a fourth optical path 68d, where the fifth light ray L55 is a part or all of the sum of the second light ray L52 and the fourth light ray L54. The first photo receiver 64 generates a first electrical signal of the pulse waveform of vascular pulsation based on the optical power of the fifth light ray L55, wherein the first electrical signal is converted into a current signal or a voltage signal.

In FIG. 11, the first fiber grating 65a or the second fiber grating 65b is adjacent to the surface of an object to be measured (such as human skin). Thus, the fiber grating adjacent to the surface of an object to be measured will shift the reflection wavelength band and the transmission wavelength band due to the vascular pulsating stress. The fiber grating, which is far away from the surface of the object to be measured, is less or not affected by the vascular pulsating stress. Therefore, the reflection wavelength band and the transmission wavelength band will only shift slightly or not shift at all. When the first fiber grating 65a or the second fiber grating 65b adjacent to the surface of an object to be measured bears the vascular pulsating stress, the area of the union of the first reflection wavelength band RB1 and the second reflection wavelength band RB2 changes with the vascular pulsating stress. In other words, the optical power of the fifth light ray L55 received by the first photo receiver 64 changes with the vascular pulsation. Thus, the optical power received by the first photo receiver 64 presents a pulse waveform signal.

In FIG. 11, the blood pressure and heart rate measuring device 60 further includes a fifth optical path 68e and a sixth optical path 68f respectively connected to the optical coupler 63 and the second fiber grating 65b. In order to avoid Fresnel reflections at the end surfaces 69a and 69b of the fifth optical path 68e and the sixth optical path 68f, there are several ways to avoid reflecting light rays back into the optical paths: (1) coating anti-reflective materials on the end surfaces 69a and 69b; (2) designing the end faces 69a and 69b to have an inclination angle greater than 3 degrees; and (3) designing the optical paths near the end faces 69a and 69b to have curled shapes with small radiuses of curvature to produce the bending loss of light.

The first optical path 68a, the second optical path 68b, the third optical path 68c, the fourth optical path 68d, the fifth optical path 68e, and the sixth optical path 68f in FIG. 11 can be fiber optical paths, waveguide optical paths, or fiber and waveguide mixed optical paths.

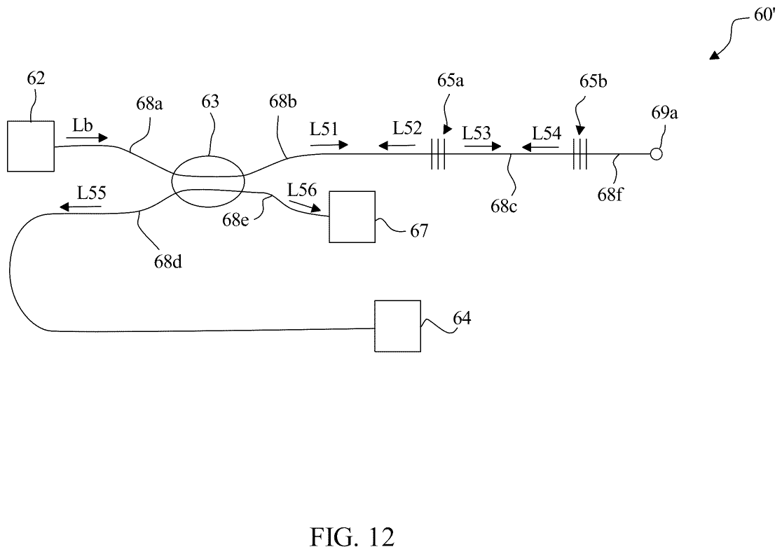

FIG. 12 is a schematic diagram showing a blood pressure and heart rate measuring device according to a tenth embodiment of the present invention. The blood pressure and heart rate measuring device of FIG. 11 is almost the same as that of FIG. 12. Their difference is that the blood pressure and heart rate measuring device 60′ of FIG. 12 further includes a second photo receiver 67 connected to the optical coupler 63 through the fifth optical path 68e. When the optical power of the broadband light source 62 is unstable, the optical power of the fifth light ray L55 received by the first photo receiver 64 will also be unstable, which may distort the finally obtained pulse waveform. The blood pressure and heart rate measuring device 60′ employs the second photo receiver 67 to improve the problem. Specifically, after the optical coupler 63 receives the broadband light Lb, the optical coupler 63 will generate a sixth light ray L56 and transmits the sixth light ray L56 to the second photo receiver 67 through the fifth optical path 68e. The second photo receiver 67 generates a second electrical signal in response to the sixth light ray L56. Since the first electrical signal and the second electrical signal both include unstable components of the broadband light source 62, the blood pressure and heart rate measuring device 60′ can cancel out the unstable components when dividing the first electrical signal by the second electrical signal. In other words, the pulsating waveform signal obtained by dividing the first electrical signal by the second electrical signal excludes the unstable component and will not be affected by the instability of the broadband light source 62.

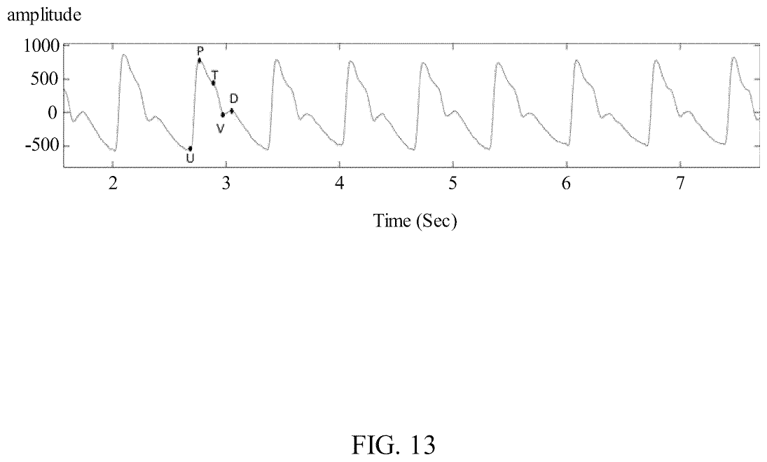

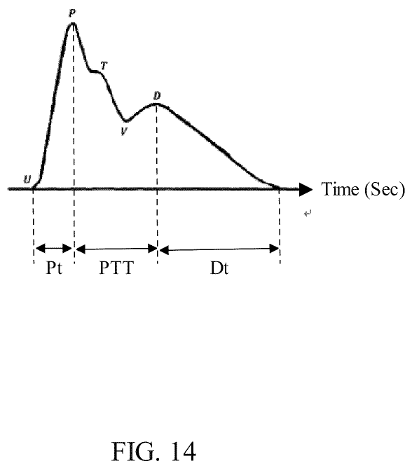

FIG. 13 is a schematic diagram showing a pulse waveform signal obtained by a blood pressure and heart rate measuring device. FIG. 14 is an enlarged view of a part of the pulse waveform signal in FIG. 13. FIG. 13 shows the filtered pulse waveform signal for retaining signal components ranging from, for example, 0.5 Hz to 12 Hz. In FIG. 13 and FIG. 14, the time interval between point P and point D is defined as PTT. The time interval from point U (i.e., an onset point) of the pulse waveform to point P (i.e., a main wave peak point) is defined as Pt. The time interval from point D (i.e., a dicrotic wave peak point) to the end point of the pulse waveform that is normally the onset point of the next pulse waveform, is defined as Dt. The heart rate can be obtained from the repetition rate of the pulse waveform signal in FIG. 13. In other words, the heart rate can be obtained from the repetition rate of point U, point P, point V (i.e., a descending wave trough point) or point D of the pulse waveform. A systolic blood pressure (SBP) and a diastolic blood pressure (DBP) can be calculated using the three parameters PTT, Pt and Dt of the pulse waveform signal and the following formula 1 and formula 2.

$$SBP = A \times ln ( PTT ) + \frac{C}{{( PTT )}^{2}} + E \times Pt + G \qquad ( 1 )$$

$$DBP = B \times ln ( PTT ) + \frac{D}{{( PTT )}^{2}} + F \times Dt + H \qquad ( 2 )$$

In formula 1 and formula 2, In is a natural logarithmic function, the value of coefficient A ranges between 300 and 900, the value of coefficient B ranges between 600 and 950, the value of coefficient C ranges between 2 and 50, the value of coefficient D ranges between 5 and between 50, the value of coefficient E ranges between 4 and 60, the value of coefficient F ranges between −25 and 50, the value of constant G ranges between 450 and 850, and the value of constant H ranges between 600 and 900.

Because the processed pulse waveform signal has multiple P-point peaks within a period of time and the three time parameters PTT, Pt and Dt provided by the pulse waveforms of these different peaks will be different, it is necessary to use the averaging method to obtain these time parameters. The averaging method is divided into two types. One type is to take out the time parameters of each pulse waveform and then average the time parameters of these pulse waveforms. Another type is to average the waveform values of these pulse waveforms at corresponding time points to obtain an average pulse waveform and then extract each time parameter from the average pulse waveform.

The deviation between the 3 dB bandwidths of the reflection spectrums of the two fiber gratings used by the blood pressure and heart rate measuring method of the present invention is less than 2 nm. The deviation between the central values of the reflection wavelengths of the reflection spectrums of the two fiber gratings used by the blood pressure and heart rate measuring method of the present invention is less than 3 nm. The broadband light source of the present invention has a 3 dB bandwidth between 2 nm and 800 nm and has a central wavelength within the wavelength range of visible light or infrared light. The broadband light source of the present invention may be, but not limited to, a light emitting diode (LED), a semiconductor optical amplifier, a semiconductor superluminescent diode, or a pumped erbium-doped fiber.

The blood pressure and heart rate measuring device of the present invention uses two fiber gratings to measure and obtain pulse waveform signals. One of the fiber gratings bears vascular pulsation to deform the fiber grating and shift its reflection wavelength band, thereby changing the optical power detected by the photo receiver to obtain a pulse waveform signal. Fiber gratings, which serve as the pulsating sensors of blood vessels, must be close to the blood vessels. However, in order to prevent the fiber grating and the adjacent fiber optical path from being damaged or broken when placing and moving the blood pressure and heart rate measuring device, the optical fiber grating and the adjacent fiber optical path are placed in a stretchable material and the fiber grating is close to the skin next to the brachial artery of the upper arm or the skin next to the radial artery of the lower arm to obtain the pulse waveform signal.

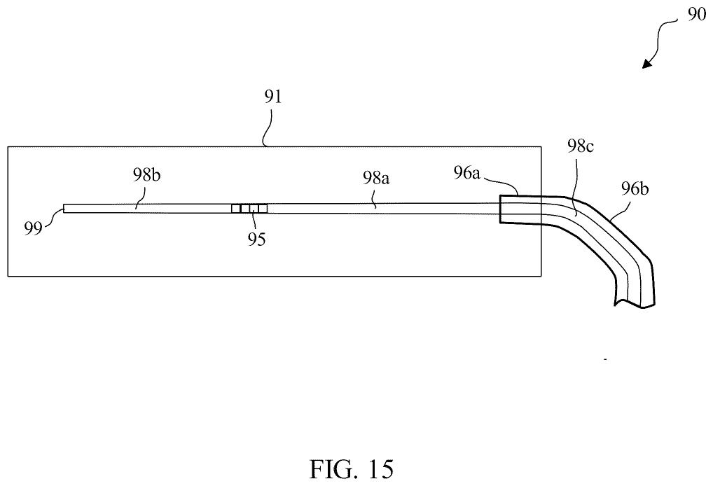

FIG. 15 is a schematic diagram showing a protective structure for protecting a fiber grating and a fiber optical path adjacent thereto. In the protective structure 90 of FIG. 15, a fiber grating 95, the adjacent fiber optical paths 98a and 98b, and a portion of the fiber optical path 98c are placed in the stretchable material 91, wherein the end face 99 of the fiber optical path 98b has an inclined angle of more than 3 degrees. Alternatively, a part of the fiber optical path in front of the end face has a bending loss of light and presents a curled state with a small radius of curvature or the end face is coated with an anti-reflective material. The fiber optical path 98c is divided into two parts respectively placed in a tube 96a and a tube 96b. The tube 96a and the fiber optical path 98c therein are placed in the stretchable material 91. The light ray can be emitted to the fiber grating 95 from the fiber optical path 98c outside the stretchable material 91 through the fiber optical path 98c and the fiber optical path 98a inside the stretchable material 91. Then, the light ray whose wavelength band is in the reflection wavelength band of the fiber grating 95 is reflected along the fiber optical paths 98a and 98c. The light ray is finally transmitted to the photo receiver to obtain the pulse waveform signal. The heart rate, the systolic blood pressure, and the diastolic blood pressure are calculated using the foregoing method.

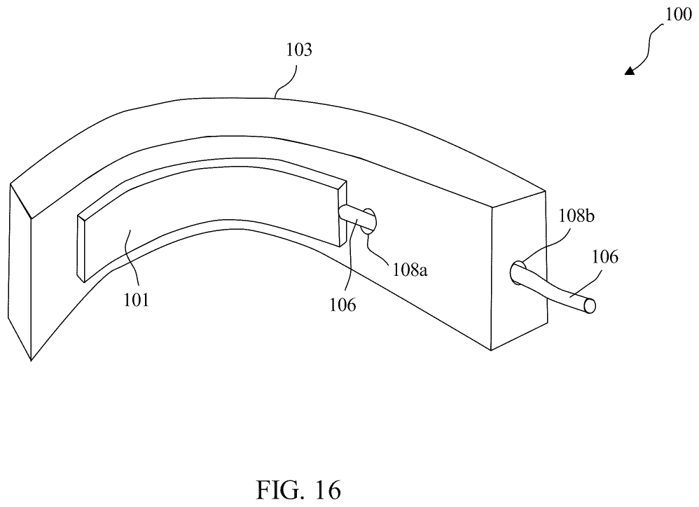

FIG. 16 is a schematic diagram showing the arm belt 100 of a blood pressure and heart rate measuring device. The arm belt 100 can surround the object to be measured (such as a human arm) to measure a blood pressure and a heart rate. In order to display the heart rate, the systolic blood pressure and the diastolic blood pressure obtained by the blood pressure and heart rate measuring device of the present invention and to conveniently measure the heart rate, the systolic blood pressure and the diastolic blood pressure, a preferred example of the present invention uses a box with a displaying function. The box has a function of displaying at least a heart rate, a systolic blood pressure, and a diastolic blood pressure. Besides, in the box there are at least a fiber, an erbium-doped fiber, an optical coupler, a semiconductor light source, an photo receiver, a power supply, a power switch, a circuit board, an integrated circuit including a heart rate/systolic blood calculation program for calculating pressure/diastolic blood pressure, a switch for selecting functional operations, and at most one fiber grating. The device inside the box is connected to the stretchable material 101 of the arm belt 100 in FIG. 16 by one strip-shaped object 106 that includes at least a fiber optical path. The purpose of the strip-shaped object 106 is to protect the fiber optical path that is connected to the fiber grating inside the stretchable material. Specifically, the strip-shaped object 106 can be the tube 96b in FIG. 15 or another flexible object covering the tube. The strip-shaped object 106 at least includes the fiber optical path 98c, as shown in FIG. 15. The stretchable material 101 is secured to one surface of a girdle 103. The internal structure of the stretchable material 101 may refer to the stretchable material 91 in FIG. 15. The girdle 103 includes holes 108a and 108b where the strip-shaped object 106 passes. Practically, all or a part of the arm belt 100 surrounds the suitable position of the arm, so that the fiber grating in the arm belt 100 can sense the pulsation of blood vessels. In another preferred example, the box also includes a module with signal transmission function.

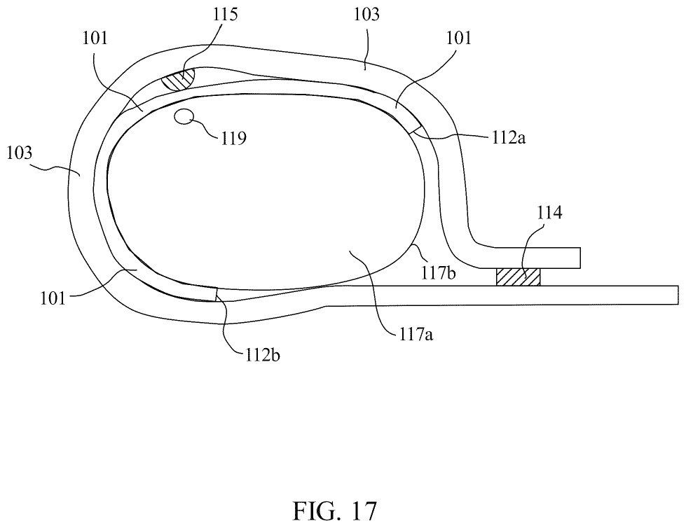

FIG. 17 is a cross-sectional view of an arm belt in FIG. 16 that surrounds an arm. In FIG. 17, a stretchable material 101 including a fiber optical path and a fiber grating is secured to one surface of the girdle 103. A force-applying body 115 is located between the stretchable material 101 and the girdle 103. The force-applying body 115 has a U-shaped or arc-shaped structure. The former has its opening facing toward the stretchable material 101 and the latter has an arc-shaped surface facing toward the stretchable material 101. When the girdle 103 surrounds an arm 117a, the force-applying body 115 applies force to the stretchable material 101, so that the stretchable material 101 closely contacts the local area of an arm skin 117b. Thus, the pulsation of the blood vessel 119 can be sensed by the fiber grating inside the stretchable material 101, wherein the fiber grating is located near the blood vessel 119. The girdle 103 has an adhesive member 114 that can join the two ends of the girdle 103. The fiber optical path in the stretchable material 101 extends from the end surface 112a or 112b of the stretchable material 101 and then passes through the girdle 103.

The embodiments described above are only to exemplify the present invention but not to limit the scope of the present invention. Therefore, any equivalent modification or variation according to the shapes, structures, features, or spirit disclosed by the present invention is to be also included within the scope of the present invention.

What is claimed is:

1. A blood pressure and heart rate measuring device comprising at least:
a broadband light source configured to provide broadband light;
an optical coupler connected to the broadband light source through a first optical path and configured to receive the broadband light;
a first fiber grating, connected to the optical coupler through a second optical path, having a first reflection wavelength band, wherein the first fiber grating is configured to receive a first light ray from the optical coupler through the second optical path and reflect a part of the first light ray whose wavelength band is within the first reflection wavelength band to generate a second light ray that returns to the optical coupler along the second optical path, and the first light ray is a part or all of the broadband light;
a second fiber grating, connected to the optical coupler through a third optical path, having a second reflection wavelength band, wherein the second fiber grating is configured to receive a third light ray from the optical coupler through the third optical path and allow a part of the third light ray whose wavelength band is not within the second reflection wavelength band to pass to generate a fourth light ray, and the third light ray is a part or all of the second light ray; and
a first photo receiver connected to the second fiber grating through a fourth optical path and configured to receive the fourth light ray from the second fiber grating through the fourth optical path and generate a first electrical signal of a pulse waveform of vascular pulsation in response to the fourth light ray;
wherein the first reflection wavelength band shifts with a vascular pulsating stress of an object to be measured.

2. The blood pressure and heart rate measuring device according to claim 1, further comprising a second photo receiver configured to receive a fifth light ray from the optical coupler through a fifth optical path and generate a second electrical signal in response to the fifth light ray, wherein the blood pressure and heart rate measuring device is configured to divide the first electrical signal by the second electrical signal to obtain a pulsating waveform signal, and the fifth light ray is a part or all of the broadband light.

3. The blood pressure and heart rate measuring device according to claim 1, wherein a deviation between 3 dB bandwidths of the first reflection wavelength band and the second reflection wavelength band is less than 2 nm and a deviation between central values of reflection wavelengths of the first reflection wavelength band and the second reflection wavelength band is less than 3 nm.

4. The blood pressure and heart rate measuring device according to claim 1, wherein a 3 dB bandwidth of the broadband light source ranges between 2 nm and 800 nm and a central wavelength of the broadband light source is within a wavelength range of visible light or infrared light.

5. A blood pressure and heart rate measuring device comprising at least:
a broadband light source configured to provide broadband light;
a first optical coupler connected to the broadband light source through a first optical path and configured to receive the broadband light, wherein the first optical coupler is configured to generate a first light ray and a second light ray in response to the broadband light and respectively transmit the first light ray and the second light ray to a second optical path and a third optical path, and the first light ray and the second light ray are parts of the broadband light;
a first fiber grating, connected to the first optical coupler through the second optical path and the third optical path, having a first reflection wavelength band, wherein the first fiber grating is configured to reflect a part of the first light ray whose wavelength band is within the first reflection wavelength band to generate a third light ray that returns to the first optical coupler along the second optical path and to reflect a part of the second light ray whose wavelength band is within the first reflection wavelength band to generate a fourth light ray that returns to the first optical coupler along the third optical path;
a second optical coupler configured to receive a fifth light ray from the first optical coupler through a fourth optical path, generate a sixth light ray in response to the fifth light ray, and transmit the sixth light ray to a fifth optical path, wherein the first optical coupler is configured to generate the fifth light ray in response to the third light ray and the fourth light ray, and the sixth light ray is a part or all of the fifth light ray;
a second fiber grating, connected to the second optical coupler through a fifth optical path, having a second reflection wavelength band, wherein the second fiber grating is configured to receive the sixth light ray from the second optical coupler through the fifth optical path and reflect a part of the sixth light ray whose wavelength band is within the second reflection wavelength band to generate a seventh light ray that returns to the second optical coupler along the fifth optical path; and
a first photo receiver configured to receive an eighth light ray from the second optical coupler through a sixth optical path and generate a first electrical signal of a pulse waveform of vascular pulsation in response to the eighth light ray;
wherein the second reflection wavelength band shifts with a vascular pulsating stress of an object to be measured.

6. The blood pressure and heart rate measuring device according to claim 5, further comprising a second photo receiver configured to receive a ninth light ray from the second optical coupler through a seventh optical path and generate a second electrical signal in response to the ninth light ray, wherein the blood pressure and heart rate measuring device is configured to divide the first electrical signal by the second electrical signal to obtain a pulsating waveform signal, and the ninth light ray is a part or all of the fifth light ray.

7. The blood pressure and heart rate measuring device according to claim 5, wherein a deviation between 3 dB bandwidths of the first reflection wavelength band and the second reflection wavelength band is less than 2 nm and a deviation between central values of reflection wavelengths of the first reflection wavelength band and the second reflection wavelength band is less than 3 nm.

8. The blood pressure and heart rate measuring device according to claim 5, wherein a 3 dB wavelength bandwidth of the broadband light source ranges between 2 nm and 800 nm and a central wavelength of the broadband light source is within a wavelength range of visible light or infrared light.

9. A blood pressure and heart rate measuring device comprising at least:
a laser light source configured to provide laser light;
a first optical coupler connected to the laser light source through a first optical path and configured to receive the laser light, wherein the first optical coupler is configured to guide the laser light to a second optical path;
a first fiber grating, connected to the first optical coupler through the second optical path, having a first reflection wavelength band and a first transmission wavelength band, wherein the laser light from the first optical coupler passes through the first fiber grating;
an Er-doped fiber connected to the first fiber grating and configured to generate broadband light that returns to the first fiber grating in response to the laser light passing through the first fiber grating;
wherein a part of the broadband light whose wavelength band is within the first transmission wavelength band passes through the first fiber grating to generate a first light ray that returns to the first optical coupler along the second optical path;
a second optical coupler configured to receive a second light ray from the first optical coupler through a third optical path, generate a third light ray in response to the second light ray, and transmit the third light ray to a fourth optical path, wherein the second light ray is a part or all of the first light ray and the third light ray is a part or all of the second light ray;
a second fiber grating, connected to the second optical coupler through the fourth optical path, having a second reflection wavelength band and a second transmission wavelength band, wherein the second fiber grating is configured to receive the third light ray from the second optical coupler through the fourth optical path and reflect a part of the third light ray whose wavelength band is within the second reflection wavelength band to generate a fourth light ray that returns to the second optical coupler along the fourth optical path; and
a first photo receiver configured to receive a fifth light ray from the second optical coupler through a fifth optical path and generate a first electrical signal of a pulse waveform of vascular pulsation in response to the fifth light ray, wherein the fifth light ray is a part or all of the fourth light ray;
wherein the second reflection wavelength band shifts with a vascular pulsating stress of an object to be measured.

10. The blood pressure and heart rate measuring device according to claim 9, further comprising a second photo receiver configured to receive a broadband light ray from the Er-doped fiber through a sixth optical path or receive a sixth light ray from the second optical coupler through a seventh optical path to generate a second electrical signal, wherein the blood pressure and heart rate measuring device is configured to divide the first electrical signal by the second electrical signal to obtain a pulsating waveform signal, and the sixth light ray is a part or all of the second light ray.

11. The blood pressure and heart rate measuring device according to claim 9, wherein a deviation between 3 dB bandwidths of the first reflection wavelength band and the second reflection wavelength band is less than 2 nm and a deviation between central values of reflection wavelengths of the first reflection wavelength band and the second reflection wavelength band is less than 3 nm.

12. A blood pressure and heart rate measuring device comprising at least:
a broadband light source configured to provide broadband light;
a first fiber grating, connected to the broadband light source through a first optical path and configured to receive the broadband light, having a first reflection wavelength band and a first transmission wavelength band, wherein a part of the broadband light whose wavelength band is within the first transmission wavelength band passes through the first fiber grating to form a first light ray;
an optical coupler connected to the first fiber grating through a second optical path and configured to receive the first light ray;
a second fiber grating, connected to the optical coupler through a third optical path, having a second reflection wavelength band and a second transmission wavelength band, wherein the second fiber grating is configured to receive a second light ray from the optical coupler through the third optical path and reflect a part of the second light ray whose wavelength band is within the second reflection wavelength band to generate a third light ray that returns to the optical coupler along the third optical path, and the second light ray is a part or all of the first light ray; and
a first photo receiver connected to the optical coupler through a fourth optical path and configured to receive a fourth light ray from the optical coupler through the fourth optical path and generate a first electrical signal of a pulse waveform of vascular pulsation in response to the fourth light ray, wherein the fourth light ray is a part or all of the third light ray;
wherein the second reflection wavelength band shifts with a vascular pulsating stress of an object to be measured.

13. The blood pressure and heart rate measuring device according to claim 12, further comprising a second photo receiver configured to receive a fifth light ray from the optical coupler through a fifth optical path and generate a second electrical signal in response to the fifth light ray, wherein the blood pressure and heart rate measuring device is configured to divide the first electrical signal by the second electrical signal to obtain a pulsating waveform signal, and the fifth light ray is a part or all of the first light ray.

14. The blood pressure and heart rate measuring device according to claim 12, wherein a deviation between 3 dB bandwidths of the first reflection wavelength band and the second reflection wavelength band is less than 2 nm and a deviation between central values of reflection wavelengths of the first reflection wavelength band and the second reflection wavelength band is less than 3 nm.

15. The blood pressure and heart rate measuring device according to claim 12, wherein a 3 dB wavelength bandwidth of the broadband light source ranges between 2 nm and 800 nm and a central wavelength of the broadband light source is within a wavelength range of visible light or infrared light.

16. A blood pressure and heart rate measuring device comprising at least:
a broadband light source configured to provide broadband light;
an optical coupler connected to the broadband light source through a first optical path and configured to receive the broadband light;
a first fiber grating, connected to the optical coupler through a second optical path, having a first reflection wavelength band and a first transmission wavelength band, wherein the first fiber grating is configured to receive a first light ray from the optical coupler through the second optical path and reflect a part of the first light ray whose wavelength band is within the first reflection wavelength band to generate a second light ray that returns to the optical coupler along the second optical path, with the first light ray being a part or all of the broadband light, and a part of the first light ray whose wavelength band is within the first transmission wavelength band passing through the first fiber grating to form a third light ray;
a second fiber grating, connected to the first fiber grating through a third optical path, having a second reflection wavelength band and a second transmission wavelength band, wherein the second fiber grating is configured to receive the third light ray from the first fiber grating through the third optical path and reflect a part of the third light ray whose wavelength band is within the second reflection wavelength band to generate a fourth light ray that returns to the optical coupler along the third optical path, the first fiber grating, and the second optical path; and
a first photo receiver connected to the optical coupler through a fourth optical path and configured to receive a fifth light ray from the optical coupler through the fourth optical path and generate a first electrical signal of a pulse waveform of vascular pulsation in response to the fifth light ray, wherein the fifth light ray is parts or all of the second light ray and the fourth light ray;
wherein the first reflection wavelength band or the second reflection wavelength band shifts with a vascular pulsating stress of an object to be measured.

17. The blood pressure and heart rate measuring device according to claim 16, further comprising a second photo receiver configured to receive a sixth light ray from the optical coupler through a fifth optical path and generate a second electrical signal in response to the sixth light ray, wherein the blood pressure and heart rate measuring device is configured to divide the first electrical signal by the second electrical signal to obtain a pulsating waveform signal, and the sixth light ray is a part or all of the broadband light.

18. A protective structure for protecting a fiber grating and a fiber optical path, comprising:
a stretchable material configured to cover the fiber grating, a first fiber optical path, a second fiber optical path and a third fiber optical path, wherein one end of the first fiber optical path is connected to the fiber grating, another end of the first fiber optical path is connected to the first portion of the third fiber optical path, and the third fiber optical path has a first portion inside the stretchable material and a second portion outside the stretchable material; and
a tube having a first portion and a second portion, wherein the first portion of the tube, located inside the stretchable material, covers the first portion of the third fiber optical path, and the second portion of the tube, located outside the stretchable material, covers the second portion of the third fiber optical path;
wherein a first light ray, entering an interior of the stretchable material from the third fiber optical path, is emitted to the fiber grating through the first fiber optical path;
wherein a part of the first light ray whose wavelength band is within a reflection wavelength band of the fiber grating is reflected to form a second light ray that returns to the first fiber optical path and then the third fiber optical path;
wherein the second light ray is received by a photo receiver to generate an electrical signal of a pulse waveform of vascular pulsation.

19. The protective structure according to claim 18, which is further secured to one surface of a girdle with a forcing-applying body located between the girdle and the stretchable material;
wherein the forcing-applying body has a U-shaped structure or an arc-shaped structure;
wherein an opening of the forcing-applying body of the U-shaped structure faces toward the stretchable material;
wherein an arc-shaped surface of the forcing-applying body of the arc-shaped structure faces toward the stretchable material;
wherein the girdle has two holes for a strip-shaped object to pass through, with the strip-shaped object being the tube or another flexible object covering the tube.

20. A blood pressure and heart rate calculating method required by a blood pressure and heart rate measuring device, comprising:
filtering a pulse waveform signal generated by vascular pulsation to retain signal components ranging from P Hz to Q Hz, thereby obtaining a filtered pulse waveform signal, where P ranges between 0.1 and 4 and Q ranges between 2 and 50;
obtaining a heart rate based on a repetition rate of an onset point, a main wave peak point, a descending wave trough point, or a dicrotic wave peak point of the filtered pulse waveform;
calculating a systolic blood pressure (SBP) and a diastolic blood pressure (DBP) using following formulas:
$$SBP = A \times ln ( PTT ) + \frac{C}{{( PTT )}^{2}} + E \times Pt + G ,$$
$$DBP = B \times ln ( PTT ) + \frac{D}{{( PTT )}^{2}} + F \times Dt + H ;$$
where ln is a natural logarithmic function, PTT is a time interval between the main wave peak point and the dicrotic wave peak point, Pt is a time interval from the onset point to the main wave peak point, Dt is a time interval from the dicrotic wave peak point to an end point of the filtered pulse waveform, a value of coefficient A ranges between 300 and 900, a value of coefficient B ranges between 600 and 950, a value of coefficient C ranges between 2 and 50, a value of coefficient D ranges between 5 and between 50, a value of coefficient E ranges between 4 and 60, a value of coefficient F ranges between −25 and 50, a value of constant G ranges between 450 and 850, and a value of constant H ranges between 600 and 900.
